(19) United States
(12) Patent Application Publication
(10) Pub. No.: US 2025/0295578 A1
(43) Pub. Date: Sep. 25, 2025

(54) METHODS AND COMPOSITIONS FOR MICROBIAL TREATMENT OF SKIN DISORDERS

(71) Applicant: IFF US HOLDING, LLC, WILMINGTON, DE (US)

(72) Inventors: Michael W Bostick, Wilmington, DE (US); Christine Brown, Newark, DE (US); Amanda Chan, Townsend, DE (US); Helene Flannery, Swedesboro, NJ (US); John Thomas Gannon, Hockessin, DE (US); Seung-Pyo Hong, Hockessin, DE (US); Mark Gerard Starr, Newark, DE (US); Rick W Ye, Wilmington, DE (US)

(21) Appl. No.: 18/254,961

(22) PCT Filed: Dec. 1, 2021
(86) PCT No.: PCT/US2021/061356

§ 371 Date: May 30, 2023

Related U.S. Application Data
(60) Provisional application No. 63/119,701, filed on Dec. 1, 2020.
(60) Provisional application No. 63/154,903, filed on Mar. 1, 2021.

(51) Int. Cl. A61K 8/99; A61Q 5/00

(52) U.S. Cl. CPC A61K 8/99; A61Q 5/006

(57) ABSTRACT

The present disclosure is directed towards skin care compositions, skin care formulations, and methods for providing treatment of scalp disorders. More specifically, the present disclosure is directed towards methods and compositions comprising a Bacillus velezensis fermentate, and/or metabolite thereof, for treating a scalp disorder, including dandruff.

### CROSS REFERENCE OF RELATED APPLICATIONS

This application is a 371 of International Application No. PCT/US21/061356, filed Dec. 1, 2021, which claims the benefit of U.S. Provisional Application No. 63/119,701, filed Dec. 1, 2020 and U.S. Provisional Application No. 63/154,903, filed Mar. 1, 2021, all of which are incorporated by reference herein in their entirety.

### FIELD OF THE DISCLOSURE

The present disclosure is directed towards skin care compositions, skin care formulations, and methods for providing treatment of scalp disorders. More specifically, the present disclosure is directed towards methods and compositions comprising a Bacillus velezensis fermentate, and/or metabolite thereof, for treating a scalp disorder, including dandruff.

### REFERENCE TO SEQUENCE LISTING SUBMITTED ELECTRONICALLY

The content of the sequence listing electronically submitted with the application as an XML file 20230523_NB41905PCT_ST25; Size: 16,610 bytes; Created: May 23, 2023) forms part of the application and is hereby incorporated herein by reference in its entirety.

### BACKGROUND

The skin functions as a barrier protecting the organism from drying out as well as protecting the organism against the penetration of external, often harmful, substances.

The skin is also home to a diverse population of microbes, the majority of which are commensal (nonpathogenic permanent residents) or transient (temporary residents) organisms. In pathogenic interactions, only the microbe benefits, while the host is eventually harmed. Many skin pathogens can be typically found living on the skin as commensal organisms, but microbial dysbiosis (or microbial imbalance), host genetic variation, and immune status may drive the transition from commensal to pathogen (Findley, K. and Grice, E. A., The Skin Microbiome: A Focus on Pathogens and Their Association with Skin Disease. PLoS Pathog. 2014, 10).

The epidermis constitutes the outermost region of the skin tissue and as such forms the actual protective sheath against the environment. The outer layer of the epidermis (Stratum corneum or Horny layer) is the part which is in contact with the environment and the particular structure of the horny layer protects the skin as well as stabilizes its own flexibility by binding a defined amount of water (P. M. Elias, Drug Dev. Res. 13, 1988, 97-105).

Spatially, the skin microbiota may extend to subepidermal compartments (Nakatsuji, T et al., The Microbiome Extends to Subepidermal Compartments of Normal Skin. Nat. Commun. 2013, 4). Regions such as the face, chest, and back, areas with a high density of sebaceous glands, promote growth of lipophilic microorganisms such as Propionibacterium and Malassezia.

Skin is a unique environment where microbes often exist as biofilms (Brandwein, et al., 2016. NPJ Biofilms Microbiomes 2:3). The biofilms can form on the epithelial surfaces of the skin or inside the follicles. In addition to cells, a biofilm consists of extracellular components such as exopolysaccharides, proteins, and DNA. This complex structure can be a physical and chemical barrier for certain compounds. But more importantly, the physiology of the microbes in the state of biofilm is very different than those in planktonic state. This is especially true for their ability to counter environmental stress and to resist various antimicrobial treatments (Koo, et al., Nature Reviews Microbiology 15:740-755, 2017).

Yeast Malassezia species isolated from both healthy and unhealthy skin have been shown to form biofilms in vitro (Angiolella, et al. 2020, Med Mycol. 0:1-7). These isolates of Malassezia globosa (M. globosa) can be highly adherent and/or hydrophobic as well as biofilm producers. Malassezia species in the form of biofilm have been shown to have a significant decrease in their susceptibility to antifungal agents (Figueredo, et al., 2016, Medical Mycology 8:863-867, 2013; Bumroogthai, et al., Medical Mycology 54:544-549). Biofilm adherence and hydrophobicity was suggested as virulence factor for Malassezia (Allen, et al., 2018, J. of Clinical & Experimental Dermatology Research 6:311, 2015; Angiolella, et al., Medical Mycology 56:110-116). Thus, strategies to remove Malassezia biofilm can be beneficial to treat various skin conditions caused by this group of organisms.

Malassezia is a predominant fungus of the skin microbiota and found on virtually everybody's scalp and implicated in the most common skin disorders such as, but not limiting to, seborrheic dermatitis, dandruff, and tinea versicolor. Dandruff is the common term for seborrhea of the scalp. It is mainly associated with Malassezia species such as Malassezia restricta (M. restricta) and Malassezia globosa (M. globosa) and has a very high prevalence of nearly 50% of the population (Schommer, N. N.; Gallo, R. L., Structure and Function of the Human Skin Microbiome. Trends Microbiol. 2013, 21, 660-668). Improvements in the disease can be achieved by therapeutic application of antifungal, but not antibacterial agents. The mechanisms underlying pathogenicity are incompletely understood. Impaired skin barrier function facilitates the course of the disease (Harding, C. Ret al., Dandruff: a condition characterized by decreased levels of intercellular lipids in scalp stratum corneum and impaired barrier function. Arch. Dermatol. Res. 2002, 294, 221-230).

Malassezia species do not have fatty acid synthase, so they have to rely on sebum lipids for carbon source. They also lack delta 2,3-enoyl-CoA isomerase for efficient unsaturated FA (e.g. oleate) utilization. Malassezia species feeds on sebum fat (by secreting a lipase or lipases that splits triglycerides into irritant fatty acids), and as sebum fat is broken down, free fatty acids (such as oleic acid) are released as by-product. Many people are sensitive to free fatty acids as they can induce hyperproliferation and scaling, or induce the release of arachidonic acid, which is also involved in inflammation, and their scalp responds by becoming irritated. In respond to the irritation, the scalp starts to become inflamed, red, and itchy, and the body shed skin cells faster than usual, in attempt to shed the irritant. The shedding of skin causes visible flakes to appear on the scalp, which is dandruff.

In addition to yeast Malassesia species, Staphylococcus species, such as S. aureus or S. epidermidis, are associated with dandruff scalp while Propionibacterial species are associated with healthy scalp (Saxena, et al., 2018, Front Cell Infect Microbiol, 8:346; Xu, et al. 2016, Sci Rep., 6: 24877. Suppressing certain populations of Staphylococcus may provide benefit for dandruff treatment. Certain Staphylococcus species including S. aureus can form biofilms (Crit Rev Microbiol, 43:602-620, 2017).

There remains a need to find methods and skin care compositions for providing treatment of scalp disorders, such as but not limiting to methods and compositions for the treatment of a dandruff condition of the scalp.

### SUMMARY

The present disclosure is directed to compositions and methods for providing treatment of scalp disorders. More specifically, the present disclosure is directed towards methods and compositions comprising a Bacillus velezensis fermentate, and/or metabolite thereof for treating a scalp disorder, including dandruff.

The inventors have unexpectedly observed that a fermentate (culture supernatant) of Bacillus velezensis not only reduces the growth of Staphylococcus species and Malassezia species but also removes biofilm of Malassezia species and Staphylococcus species.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder.

In one aspect the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder, wherein the composition reduces the growth of Staphylococcus species.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder, wherein the composition reduces the growth of Malassezia species.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder, wherein the composition removes biofilm of Malassezia and Staphylococcus species.

In one embodiment, the composition is a skin care product comprising an effective amount of a skin care composition for use in the treatment of a scalp disorder and one or more dermatologically or skin care acceptable component, wherein said skin care composition comprises an effective amount of a Bacillus velezensis fermentate, and/or metabolite thereof, wherein said skin care product reduces and/or treats said scalp disorder.

In one embodiment, the composition is a skin care product selected from the group consisting of a lotion, a serum, a jelly, a cream, a gel, an emulsion, a mask, a patch, or a stick comprising one or more dermatologically or skin care acceptable components and at least about 1%, 2%, 3%, 4%, 5%, 6%, 7%, 8%, 9% up to 10% of the skin care composition (comprising an effective amount of a Bacillus velezensis fermentate, and/or metabolite thereof) described herein on a weight basis relative to a total weight of said skin care formulation.

In some embodiments provided herein, the composition comprises an effective amount of a Bacillus velezensis fermentate, and/or a Bacillus velezensis cell lysate, and/or a Bacillus velezensis fraction, and/or a Bacillus velezensis metabolite, for treating a scalp disorder, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one embodiment, the method is a method for treating a scalp disorder in a subject in need thereof, comprising administering an effective amount of a Bacillus velezensis fermentate, and/or metabolite thereof, to said subject.

In one embodiment, the method is a method for treating and/or reducing a dandruff condition of the scalp in a subject in need thereof, comprising administering an effective amount of a Bacillus velezensis fermentate, and/or metabolite thereof, to said subject.

In some embodiments provided herein, the method comprises administering an effective amount of a Bacillus velezensis fermentate, and/or a Bacillus velezensis cell lysate, and/or a Bacillus velezensis fraction, and/or a Bacillus velezensis metabolite, for treating a scalp disorder, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one embodiment, the method is a method for treating and/or reducing a dandruff condition of the scalp in a subject in need thereof, comprising topically administering a skin care product comprising a skin care composition described herein to said subject.

Further provided herein is a kit, comprising the compositions described herein and instructions for the use thereof to treat a skin condition. In some embodiments, the kit further comprises one or more applicator configured to apply the composition.

### DETAILED DESCRIPTION

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. It will be understood that in the following, embodiments referred to in relation to one broad aspect of the invention are equally applicable to each of the other broad aspects of the present invention described above. It will be further understood that, unless the context dictates otherwise, the embodiments described below may be combined.

### Scalp Disorders

For the purpose of the present disclosure, the term “scalp disorder” includes a dandruff condition of the scalp (seborrheic dermatitis), unbalanced ecoflora of the scalp, discomfort of the scalp, tinea versicolor, dry skin, irritated skin, or any one combination thereof.

For the purpose of the present disclosure, the term “dandruff condition” refers to a condition manifested by a scalp presenting excessive dryness or excessive secretion of sebum, which, depending on the case, may be characterized by the presence of dry or greasy or oily dandruff, or even pruritis and/or an inflammation of the epidermis.

Dry dandruff conditions reflect a xerosis of the scalp, which may be combined with excessively rapid renewal of its stratum corneum. Dry dandruff flakes are generally in the form of small white or grey flakes and are spread over the scalp and on the clothing, giving rise to an unaesthetic visual effect.

The itching associated with dryness of the scalp may lead to erythema, pruritus or even inflammation.

Greasy or oily dandruff conditions are one of the forms of seborrhoeic dermatitis. Individuals suffering from seborrhoeic dermatitis have an erythematous scalp covered with large, greasy or oily, yellow scales which accumulate so as to form packets. They have a pruritic scalp, and often have burning sensations on the affected areas. These phenomena may be amplified by the presence of pathogenic microorganisms, especially Malassezia species (Malassezia spp.). These microorganisms having the property of releasing fatty acids from the sebum may impair the barrier function of the epidermis and give rise to inflammation.

During dandruff conditions of the scalp, the cutaneous barrier is unbalanced, its integrity and its hydration are impaired, and its ecoflora is disturbed. The skin of the scalp is irritated and pruritic, brittle, less hydrated, and sensitive to infections.

In addition to yeast Malassesia species, Staphylococcus species, such as S. aureus or S. epidermidis, are associated with dandruff scalp while Propionibacterial species are associated with healthy scalp (Saxena, et al., 2018, Front Cell Infect Microbiol, 8:346; Xu, et al. 2016, Sci Rep., 6: 24877. Suppressing certain populations of Staphylococcus may provide benefit for dandruff treatment. Certain Staphylococcus species including S. aureus can form biofilms (Crit Rev Microbiol, 43:602-620, 2017).

Unexpectedly, the inventors have observed that a fermentate (culture supernatant) of Bacillus velezensis can reduce the growth of Staphylococcus species and Malassezia species. Furthermore, the inventors have unexpectedly observed that a fermentate (culture supernatant) of Bacillus velezensis removes biofilm of Malassezia species and Staphylococcus species, thereby making it possible to reduce dandruff conditions in subjects in need thereof.

Malassezia species described herein include, but are not limited to, Malassezia restricta (M. restricta), Malassezia globosa (M. globosa), Malassezia furfur (M. furfur), Malassezia sympodialis (M. sympodialis), Malassezia phylotype 5 (M. phylotype 5), other uncharacterized Malassezia species, and any combination thereof.

Staphylococcus species described herein include, but are not limited to, Staphylococcus aureus, Staphylococcus epidermidis, Staphylococcus haemolyticus, Staphylococcus capitis, other uncharacterized Staphylococcus species, and any combination thereof.

### Microorganisms, Fractions Thereof, Cell-Lysates Thereof, Fermentates Thereof and Metabolites Thereof

As used herein, “microorganism” or “microbe” refers to a bacterium, a fungus, a virus, a protozoan, and other microbes or microscopic organisms.

In some embodiments, the Bacillus velezensis (B. velezensis) cells can be subjected to treatments that render them non-replicating, for example, exposure to heat, desiccation, γ-irradiation, or UV-irradiation. A non-replicating B. velezensis can be a dead cell or a living cell that has been rendered incapable of cell division. A non-replicating B. velezensis can be an intact cell or a cell that has undergone partial or complete lysis. In some embodiments, the non-replicating cells can include a mixture of intact and lysed cells.

As used herein, the term “probiotic” or “probiotic microorganism” are used interchangeably herein and refer to a live microorganism (including bacteria or yeasts for example) which, when administered (topically or orally) in sufficient amounts, beneficially affects the host organism, i.e. by conferring one or more demonstrable benefits, such as a reduced dandruff condition, on the host organism.

In one aspect the microorganism suitable for use in the present invention includes strains of Bacillus velezensis.

In some aspects the microorganism suitable for use in the present invention includes a bacterial strain having a 16S ribosomal RNA sequence displaying at least 97.0% sequence similarity to a 16S ribosomal RNA sequence of B. velezensis E04 (SEQ ID NO: 6) deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 (SEQ ID NO: 7) deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 (SEQ ID NO: 8) deposited at Westerdijk Fungal Biodiversity Institute (WFDB) under number CBS147471, and any one combination thereof.

The 16S ribosomal RNA sequence of Bacillus velezensis E04 is as follows:

(SEQ ID NO: 6)
CGAUGCGUAGCCGACCUGAGAGGGUGAUCGGCCACACUGGGACUGA
GACACGGCCCAGACUCCUACGGGAGGCAGCAGUAGGGAAUCUUCCGCAAU
GGACGAAAGUCUGACGGAGCAACGCCGCGUGAGUGAUGAAGGUUUUCGGA
UCGUAAAGCUCUGUUGUUAGGGAAGAACAAGUGCCGUUCAAAUAGGGCGG
CACCUUGACGGUACCUAACCAGAAAGCCACGGCUAACUACGUGCCAGCAGC
CGCGGUAAUACGUAGGUGGCAAGCGUUGUCCGGAAUUAUUGGGCGUAAAG
GGCUCGCAGGCGGUUUCUUAAGUCUGAUGUGAAAGCCCCCGGCUCAACCG
GGGAGGGUCAUUGGAAACUGGGGAACUUGAGUGCAGAAGAGGAGAGUGGA
AUUCCACGUGUAGCGGUGAAAUGCGUAGAGAUGUGGAGGAACACCAGUGG
CGAAGGCGACUCUCUGGUCUGUAACUGACGCUGAGGAGCGAAAGCGUGGG
GAGCGAACAGGAUUAGAUACCCUGGUAGUCCACGCCGUAAACGAUGAGUGC
UAAGUGUUAGGGGGUUUCCGCCCCUUAGUGCUGCAGCUAACGCAUUAAGC
ACUCCGCCUGGGGAGUACGGUCGCAAGACUGAAACUCAAAGGAAUUGACG
GGGGCCCGCACAAGCGGUGGAGCAUGUGGUUUAAUUCGAAGCAACGCGAA
GAACCUUACCAGGUCUUGACAUCCUCUGACAAUCCUAGAGAUAGGACGUCC
CCUUCGGGGGCAGAGUGACAGGUGGUGCAUGGUUGUCGUCAGCUCGUGU
CGUGAGAUGUUGGGUUAAGUCCCGCAACGAGCGCAACCCUUGAUCUUAGU
UGCCAGCAUUCAGUUGGGCACUCUAAGGUGACUGCCGGUGACAAACCGGA
GGAAGGUGGGGAUGACGUCAAAUCAUCAUGCCCCUUAUGACCUGGGCUAC
ACACGUGCUACAAUGGACAGAACAAAGGGCAGCGAAACCGCGAGGUUAAGC
CAAUCCCACAAAUCUGUUCUCAGUUCGGAUCGCAGUCUGCAACUCGACUGC
GUGAAGCUGGAAUCGCUAGUAAUCGCGGAUCAGCAUGCCGCGGUGAAUAC
GUUCCCGGGCCUUGUACACACCGCCCGUCACACCACGAGAGUUUGUAACAC
CCGAAGUCGGUGAGGUAACCUUUUAGGAGCCAGCCGCCGAAGGUGGGACA
GAUGAUUGGGGUGAAGUCGUAACAAGGUAGCCGUAUCGGAAGGUGCGGCU
GGAUCACCUCCUUU.

The 16S ribosomal RNA sequence of Bacillus velezensis H02 is as follows:

(SEQ ID NO: 7)
CGAUGCGUAGCCGACCUGAGAGGGUGAUCGGCCACACUGGGACUGA
GACACGGCCCAGACUCCUACGGGAGGCAGCAGUAGGGAAUCUUCCGCAAU
GGACGAAAGUCUGACGGAGCAACGCCGCGUGAGUGAUGAAGGUUUUCGGA
UCGUAAAGCUCUGUUGUUAGGGAAGAACAAGUGCCGUUCAAAUAGGGCGG
CACCUUGACGGUACCUAACCAGAAAGCCACGGCUAACUACGUGCCAGCAGC
CGCGGUAAUACGUAGGUGGCAAGCGUUGUCCGGAAUUAUUGGGCGUAAAG
GGCUCGCAGGCGGUUUCUUAAGUCUGAUGUGAAAGCCCCCGGCUCAACCG
GGGAGGGUCAUUGGAAACUGGGGAACUUGAGUGCAGAAGAGGAGAGUGGA
AUUCCACGUGUAGCGGUGAAAUGCGUAGAGAUGUGGAGGAACACCAGUGG
CGAAGGCGACUCUCUGGUCUGUAACUGACGCUGAGGAGCGAAAGCGUGGG
GAGCGAACAGGAUUAGAUACCCUGGUAGUCCACGCCGUAAACGAUGAGUGC
UAAGUGUUAGGGGGUUUCCGCCCCUUAGUGCUGCAGCUAACGCAUUAAGC
ACUCCGCCUGGGGAGUACGGUCGCAAGACUGAAACUCAAAGGAAUUGACG
GGGGCCCGCACAAGCGGUGGAGCAUGUGGUUUAAUUCGAAGCAACGCGAA
GAACCUUACCAGGUCUUGACAUCCUCUGACAAUCCUAGAGAUAGGACGUCC
CCUUCGGGGGCAGAGUGACAGGUGGUGCAUGGUUGUCGUCAGCUCGUGU
CGUGAGAUGUUGGGUUAAGUCCCGCAACGAGCGCAACCCUUGAUCUUAGU
UGCCAGCAUUCAGUUGGGCACUCUAAGGUGACUGCCGGUGACAAACCGGA
GGAAGGUGGGGAUGACGUCAAAUCAUCAUGCCCCUUAUGACCUGGGCUAC
ACACGUGCUACAAUGGACAGAACAAAGGGCAGCGAAACCGCGAGGUUAAGC
CAAUCCCACAAAUCUGUUCUCAGUUCGGAUCGCAGUCUGCAACUCGACUGC
GUGAAGCUGGAAUCGCUAGUAAUCGCGGAUCAGCAUGCCGCGGUGAAUAC
GUUCCCGGGCCUUGUACACACCGCCCGUCACACCACGAGAGUUUGUAACAC
CCGAAGUCGGUGAGGUAACCUUUUAGGAGCCAGCCGCCGAAGGUGGGACA
GAUGAUUGGGGUGAAGUCGUAACAAGGUAGCCGUAUCGGAAGGUGCGGCU
GGAUCACCUCCUUU.

The 16S ribosomal RNA sequence of Bacillus velezensis F03 is as follows:

(SEQ ID NO: 8)
CGAUGCGUAGCCGACCUGAGAGGGUGAUCGGCCACACUGGGACUGAGACA
CGGCCCAGACUCCUACGGGAGGCAGCAGUAGGGAAUCUUCCGCAAUGGAC
GAAAGUCUGACGGAGCAACGCCGCGUGAGUGAUGAAGGUUUUCGGAUCGU
AAAGCUCUGUUGUUAGGGAAGAACAAGUGCCGUUCAAAUAGGGCGGCACCU
UGACGGUACCUAACCAGAAAGCCACGGCUAACUACGUGCCAGCAGCCGCGG
UAAUACGUAGGUGGCAAGCGUUGUCCGGAAUUAUUGGGCGUAAAGGGCUC
GCAGGCGGUUUCUUAAGUCUGAUGUGAAAGCCCCCGGCUCAACCGGGGAG
GGUCAUUGGAAACUGGGGAACUUGAGUGCAGAAGAGGAGAGUGGAAUUCC
ACGUGUAGCGGUGAAAUGCGUAGAGAUGUGGAGGAACACCAGUGGCGAAG
GCGACUCUCUGGUCUGUAACUGACGCUGAGGAGCGAAAGCGUGGGGAGCG
AACAGGAUUAGAUACCCUGGUAGUCCACGCCGUAAACGAUGAGUGCUAAGU
GUUAGGGGGUUUCCGCCCCUUAGUGCUGCAGCUAACGCAUUAAGCACUCC
GCCUGGGGAGUACGGUCGCAAGACUGAAACUCAAAGGAAUUGACGGGGGC
CCGCACAAGCGGUGGAGCAUGUGGUUUAAUUCGAAGCAACGCGAAGAACCU
UACCAGGUCUUGACAUCCUCUGACAAUCCUAGAGAUAGGACGUCCCCUUCG
GGGGCAGAGUGACAGGUGGUGCAUGGUUGUCGUCAGCUCGUGUCGUGAG
AUGUUGGGUUAAGUCCCGCAACGAGCGCAACCCUUGAUCUUAGUUGCCAG
CAUUCAGUUGGGCACUCUAAGGUGACUGCCGGUGACAAACCGGAGGAAGG
UGGGGAUGACGUCAAAUCAUCAUGCCCCUUAUGACCUGGGCUACACACGU
GCUACAAUGGACAGAACAAAGGGCAGCGAAACCGCGAGGUUAAGCCAAUCC
CACAAAUCUGUUCUCAGUUCGGAUCGCAGUCUGCAACUCGACUGCGUGAAG
CUGGAAUCGCUAGUAAUCGCGGAUCAGCAUGCCGCGGUGAAUACGUUCCC
GGGCCUUGUACACACCGCCCGUCACACCACGAGAGUUUGUAACACCCGAAG
UCGGUGAGGUAACCUUUUAGGAGCCAGCCGCCGAAGGUGGGACAGAUGAU
UGGGGUGAAGUCGUAACAAGGUAGCCGUAUCGGAAGGUGCGGCUGGAUCA
CCUCCUUU.

As used herein, the term “fraction” or “fraction of Bacillus velezensis” or ““fraction of the at least one microorganism of Bacillus velezensis” or “fraction thereof” more particularly denotes a fragment of the said microorganism, which has efficacy in the treatment of dandruff conditions of the scalp by analogy with the said whole microorganism. A fraction of the at least one Bacillus velezensis organism includes metabolites (also referred to as Bacillus velezensis metabolites) obtained from said Bacillus velezensis. In one aspect the fraction of the Bacillus velezensis comprises one or more metabolite(s) (active compound(s), also referred to as bioactives, derived from the metabolism of a Bacillus velezensis microorganism and also having efficacy in the treatment of a scalp disorder.

As used herein, the term “metabolite(s)” or “metabolite(s) of the at least one Bacillus velezensis” or “metabolite(s) of Bacillus velezensis” or “metabolite(s) thereof” or “Bacillus velezensis metabolite(s)” or “metabolite actives” or “bioactive” are used interchangeably and refer to any substance derived from the metabolism of a Bacillus velezensis microorganism and also having efficacy in the treatment of a scalp disorder. In one aspect, the one or more metabolite(s) were produced during the culture (fermentation) of the least one Bacillus velezensis for use in the treatment of a scalp disorder. These metabolites of Bacillus velezensis for use in the treatment of a scalp disorder include, but are not limited to, primary metabolites (metabolites directly involved in normal growth, development and reproduction), soluble metabolites, fatty acids, sugars, sugar alcohols, peptides, proteins, nucleotides, secondary metabolites, polynucleotides and polysaccharides.

It will be apparent that the fraction may be used directly in the compositions and formulations of the present invention, or that one or more of the bioactive (metabolites) may be isolated form the fraction by any suitable means prior to use.

In one aspect, the Bacillus velezensis metabolites and/or fractions that are suitable for use in the invention may be administered in the form of a lysate.

As used herein, the term “cell lysate” or “lysate” refers to cells which have been lysed by any suitable means. The term “cell lysate” or “lysate” conventionally denotes a material obtained after the destruction or dissolution of biological cells via a phenomenon known as cell lysis, thus giving rise to the release of the intracellular biological constituents naturally contained in the cells of the microorganism under consideration. For the purposes of the present disclosure, the term “lysate” is used without preference to denote the whole lysate obtained via lysis of the microorganism under consideration or only a fraction thereof. The lysate used is thus totally or partially formed from the intracellular biological constituents and from the constituents of the cell walls and membranes. Advantageously, a lysate used for the invention may be the whole lysate obtained via lysis of the microorganism under consideration. This cell lysis may be accomplished by any suitable means, such as but not limiting to, an osmotic shock, a heat shock, ultrasonication, sonication, homogenization, shearing, chemical lysis or under a mechanical stress of centrifugation type.

In one aspect the cell lysate of Bacillus velezensis comprises one or more metabolite(s) (active compounds, also referred to as bioactive, derived from the metabolism of a Bacillus velezensis microorganism and also having efficacy in the treatment of a scalp disorder.

It will be apparent that the cell lysate may be used directly in the compositions and formulations of the present invention, or that one or more of the bioactive (metabolites) may be isolated form the cell lysate by any suitable means prior to use.

A lysate may be used in various forms, in the form of a solution or in a pulverulent form. The microorganism(s) may be included in a composition according to the invention in live, semi-active or inactivated or dead form.

For the purposes of the invention, an “inactivated” or “dead” microorganism is a microorganism that is no longer capable of forming colonies in cultures. The dead or inactivated microorganisms may have intact or broken cell membranes.

The dead or inactivated microorganisms may be obtained via any method known to those skilled in the art.

In some embodiments, the cell debris is removed prior to use. In one embodiment the cell lysates are filtered prior to use. In one embodiment, the cells are lysed by, for example sonication, homogenization, shearing or chemical lysis.

As used herein, the term “fermentate” is to be understood as a composition for which one or more living microbial strains have been propagated in a nutrient medium. In one aspect, “fermentate” refers to the supernatant of a cell culture of at least one Bacillus velezensis strain from which the cells have been removed. In one embodiment the cells are removed by centrifugation, optionally followed by filter sterilization. In one embodiment the fermentate (supernatant of a cell culture) is obtained by filtration of the culture medium in which Bacillus velezensis cells were cultivated.

In one aspect, “fermentate” refers to the supernatant of a cell culture of Bacillus velezensis selected from the group of a fermenatate of B. velezensis E04, a fermentate of B. velezensis H02, a fermentate of B. velezensis F03, or any one combination thereof.

In one aspect the fermentate of at least one Bacillus velezensis strain comprises one or more metabolite(s) (active compounds, also referred to as bioactive) derived from the metabolism of a Bacillus velezensis strain and also having efficacy in the treatment of a scalp disorder.

It will be apparent that the fermentate may be used directly in the compositions and formulations of the present invention, or that one or more of the bioactive (metabolites) may be isolated form the fermentate by any suitable means prior to use.

In one aspect the fermentate may comprise one or more bioactive with a molecular weight of less than 50 kDa, that were produced during the fermentation of at least one Bacillus velezensis strain, wherein said bioactive reduce the growth of Malassezia species.

In one aspect the fermentate may comprise one or more bioactive with a molecular weight of less than 30 kDa, that were produced during the fermentation of at least one Bacillus velezensis strain, wherein said bioactive reduce the growth of Malassezia species.

In one aspect the fermentate may comprise one or more bioactive with a molecular weight of less than 10 kDa, that were produced during the fermentation of at least one Bacillus velezensis strain, wherein said bioactive reduce the growth of Malassezia species.

In one aspect the fermentate may comprise one or more bioactive with a molecular weight of less than 3 kDa, that were produced during the fermentation of at least one Bacillus velezensis strain, wherein said bioactive reduce the growth of Malassezia species.

In one aspect the fermentate may comprise one or more metabolites, such as but not limiting to soluble metabolites, that were produced during the fermentation of at least one Bacillus velezensis strain.

In one embodiment a fermentate originating from the culture (fermentation) of Bacillus velezensis may be used in the methods and/or uses of the present invention.

The nutrient medium used for preparing the fermentate is any medium comprising necessary nutrients suitable for propagating selected microorganisms. Suitable nutrients include but are not limited to amino peptides, peptides, yeast extract and/or vitamins. The medium can be based on dairy products, such as milk, cereals, fruits and/or vegetables.

As used herein, the term “soluble metabolite” refers to a metabolite or metabolites present in the supernatant of a cell culture from which the cells have been removed. In one embodiment the culture is grown to a cell density of at least about OD600 0.5. In one embodiment the cells are removed by centrifugation. In one embodiment the supernatant is filtered. It will be apparent that the supernatant may be used directly in the formulations of the present invention, or that one or more of the metabolites may be isolated form the supernatant by any suitable means prior to use.

In some embodiments, the compositions of the invention can include Bacillus velezensis fermentates, from which all or substantially all, of the Bacillus velezensis cells have been removed. Methods for separating cells from growth media are well known in the art and can rely upon physical methods, for example, centrifugation to produce a cell pellet and a culture supernatant, filtration, ultrafiltration, tangential flow-filtration, normal flow filtration or reverse osmosis. Alternatively, or in addition, the separation method can be ligand-based and include, for example, an antibody that specifically binds to Bacillus velezensis. The antibody can be coupled to a solid support such as a magnetic bead.

In some embodiments, the compositions of the invention include Bacillus velezensis strains that are partially or substantially isolated from the media in which they were grown and inactivated. The Bacillus velezensis strains can be non-replicating, e.g., inactivated, for example, by heat-treatment. The cells can be lyophilized or freeze-dried under conditions that preserve cell viability. Methods of lyophilizing are well known in the art.

In one embodiment the fermentate may comprise Bacillus velezensis strains consisting essentially of nonviable cells (e.g. intact cells).

The term “consisting essentially of” in the context of the fermentate includes that at least 90% of Bacillus velezensis have the indicated property (e.g. intact non-viable cells). Suitably at least 95% have the indicated property. Suitably at least 97% have the indicated property. Suitably at least 99% have the indicated property. In some embodiments at least 100% have the indicated property.

A “cell-free fermentate” (synonymous to the term “fermentation supernatant” or “cell-free cell culture supernatant”) as used herein means that the fermentate is substantially free of viable Bacillus velezensis cells.

In one embodiment, the composition is a skin care composition for use in the treatment of a scalp disorder, comprising an effective amount of a fermentation supernatant from a fermentation of at least one Bacillus velezensis strain, wherein said composition reduces and/or treats said scalp disorder.

In one embodiment, the fermentate for use in the compositions and methods and/or uses of the present invention may be substantially free of viable Bacillus velezensis cells, typically containing zero (or substantially) viable cells/mL fermentate.

### Skin Care Compositions and Skin Care Products for the Treatment of Skin Disorders Such as a Dandruff Condition.

As used herein the term “skin care composition” refers to a composition comprising at least one skin care benefit agent capable of providing a skin care benefit.

As used herein the term “skin care benefit agent” or “active agent” or “bioactive” are used interchangeably, and refer to a Bacillus velezensis fermentate, and/or a Bacillus velezensis cell lysate, and/or a Bacillus velezensis fraction, and/or a Bacillus velezensis metabolite, that can provide a skin care benefit.

In some embodiments provided herein, the skin care benefit agent includes an effective amount of a Bacillus velezensis fermentate, and/or a Bacillus velezensis cell lysate, and/or a Bacillus velezensis fraction, and/or a Bacillus velezensis metabolite, for treating a scalp disorder, wherein said Bacillus velezensis is selected from the group consisting of a bacterial strain having a 16S ribosomal RNA sequence displaying at least 97.0% sequence similarity to a 16S ribosomal RNA sequence of B. velezensis E04 (SEQ ID NO: 6) deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 (SEQ ID NO: 7) deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 (SEQ ID NO: 8) deposited at Westerdijk Fungal Biodiversity Institute (WFDB) under number CBS147471, and any one combination thereof.

As used herein the term “skin care benefit” refers to a benefit provided by an active agent (or skin care composition and/or skin care product comprising an effective amount of said active agent) when applied topically to a skin. In one aspect of the invention the skin care benefit is selected from the group consisting of preventing a dandruff condition, reducing a dandruff condition, treatment of a dandruff condition, reducing the occurrence of Malassezia species and/or Staphylococcus species on the skin (scalp), removing biofilm formation of Malassezia species and/or Staphylococcus species on the skin (scalp), preventing or reducing biofilm formation of Malassezia species and/or Staphylococcus species on the skin (scalp), improving the barrier function of the skin, skin moisturizing (protecting the skin against dehydration by maintaining, restoring and/or strengthening the moisturization of the skin) or any one combination thereof.

As used herein, the term “biofilm” refers to a community of microorganisms embedded in an extracellular polymer matrix attached to a surface. The extracellular polymer matrix is a polymeric conglomeration generally composed of extracellular DNA, proteins, and polysaccharides. A biofilm may have one or more microorganisms and further includes water and may include other trapped particles. The microorganisms may be gram positive or gram-negative bacteria (aerobic or anaerobic); algae, protozoa, and/or yeast or filamentous fungi. In one embodiment, the biofilm is living cells including one or more Malassezia species.

As used herein, “surface” means any structure having sufficient mass to allow for attachment of biofilm. A surface includes a hard surface and a soft surface. Hard surfaces include, but are not limited to metal, glass, ceramics, wood, minerals (rock, stone, marble, granite), aggregate materials such as concrete, plastics, composite materials, hard rubber materials, and gypsum. Other surfaces may be biological surfaces, such as skin, scalp, or keratin.

Additional benefit agents for skin care can include antidandruff active agents.

Examples of antidandruff active agents include keratolytic agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones (e.g. ciclopirox), naturopathic agents such as Melaleuca sp. oil, Aloe vera, and probiotic microorganisms. (Indian J. Dermatol, 2010 April-June; 55(2): 130-134).

Skin care benefit agents of a Bacillus velezensis fermentate, and/or a Bacillus velezensis cell lysate, and/or a Bacillus velezensis fraction, and/or a Bacillus velezensis metabolite, further include agents (actives) that reduced growth of Malassezia species and/or Staphylococcus species, and agents that removes biofilm of Malassezia species and/or Staphylococcus species.

In one aspect, the skin care benefit agent is a bioactive agent selected from the group consisting of a bioactive agent with a molecular weight of less than 50 kDa, a bioactive agent with molecular weight of less than 30 kDa, a bioactive agent with molecular weight of less than 10 kDa, a bioactive agent with molecular weight of less than 3 kDa less and any one combination thereof.

In one aspect, the skin care benefit agent is a bioactive agent with a molecular weight of less than 50 kDa.

In one aspect, the skin care benefit agent is a bioactive agent with a molecular weight of less than 30 kDa.

In one aspect, the skin care benefit agent is a bioactive agent with a molecular weight of less than 10 kDa.

In one aspect, the skin care benefit agent is a bioactive agent with a molecular weight of less than 3 kDa.

In one aspect, the skin care benefit agent (active agent) consisting of a Bacillus velezensis fermentate, and/or a fraction thereof, is formulated in a skin care composition

It will be understood that the skin care composition for use in the present invention may comprise at least one fermenate of Bacillus velezensis, and/or at least one metabolite of Bacillus velezensis.

It will be further apparent that the skin care composition for use according to the present invention may comprise, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0 about 45.0%, about 50.0% by weight of the Bacillus velezensis fermentate(s), and/or metabolite thereof.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder.

In one aspect the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder, wherein the composition reduces the growth of Staphylococcus species.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder, wherein the composition reduces the growth of Malassezia species.

In one embodiment, the composition is a skin care composition for use in the treatment of a scalp disorder, comprising an effective amount of a Bacillus velezensis fermentate, and/or metabolite thereof, wherein said composition reduces and/or treats said scalp disorder, wherein the composition removes biofilm of Malassezia and Staphylococcus species.

In one embodiment, the composition is a skin care product comprising an effective amount of a skin care composition for use in the treatment of a scalp disorder, comprising an effective amount of a Bacillus velezensis fermentate, and/or metabolite thereof, wherein said composition reduces and/or treats said scalp disorder, and one or more dermatologically or skin care acceptable component.

In some embodiments provided herein, the skin care composition or skin care product comprises an effective amount of a Bacillus velezensis fermentate, and/or a Bacillus velezensis cell lysate, and/or a Bacillus velezensis fraction, and/or a Bacillus velezensis metabolite, for treating a scalp disorder, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 50 kDa, wherein said composition reduces and/or treats said scalp disorder.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 50 kDa, wherein said composition reduces growth of Malassezia species.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 30 kDa, wherein said composition reduces and/or treats said scalp disorder.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 30 kDa, wherein said composition reduces growth of Malassezia species.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 10 kDa, wherein said composition reduces and/or treats said scalp disorder.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 10 kDa, wherein said composition reduces growth of Malassezia species.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 3 kDa, wherein said composition reduces and/or treats said scalp disorder.

In one embodiment, the composition is a skin care composition for use in the treatment of a scalp disorder, comprising an effective amount of a Bacillus velezensis fermentate, wherein said Bacillus velezensis fermentate comprises one or more bioactive with a molecular weight of less than 3 kDa, wherein said composition reduces growth of Malassezia species.

In one embodiment, the composition is a skin care product selected from the group consisting of a lotion, a serum, a jelly, a cream, a gel, an emulsion, a mask, a patch, or a stick comprising one or more dermatologically or skin care acceptable components and at least about 1%, 2%, 3%, 4%, 5%, 6%, 7%, 8%, 9% up to 10% of the skin care composition (comprising an effective amount of a Bacillus velezensis fermentate, and/or metabolite thereof) described herein on a weight basis relative to a total weight of said skin care formulation.

In some embodiments, the compositions of the invention may include Bacillus velezensis fermentate, and/or metabolite thereof, in combination with one or more dermatologically or skin care acceptable component carrier.

The Bacillus velezensis can be non-replicating, e.g., inactivated, for example, by heat-treatment. The dosage may vary, but can range from the equivalent of about 10² to about 10¹² cfu/g, e.g., 1×10² cfu/g, 5×10² cfu/g, 1×10³ cfu/g, 5×10³ cfu/g, 1×10⁴ cfu/g, 5×10⁴ cfu/g, 1×10⁵ cfu/g, 5×10⁵ cfu/g, 1×10⁶ cfu/g, 5×10⁶ cfu/g, 1×10⁷ cfu/g, 5×10⁷ cfu/g, 1×10⁸ cfu/g, 5×10⁴ cfu/g, 1×10⁹ cfu/g, 5×10⁹ cfu/g, 1×10¹⁰ cfu/g, 5×10¹⁰ cfu/g, 1×10¹¹ cfu/g, 5×10¹¹ cfu/g, 1×10¹² cfu/g of dry weight.

In some embodiments, the Bacillus velezensis fermentate, and/or metabolite thereof, can be sterilized using conventional sterilization techniques before or after it is combined with the one or more dermatologically or skin care acceptable component

In one embodiment, the skin care composition is formulated in a skin care product/formulation for administration to the skin.

It will be further understood that the skin care composition for use in the present invention may further comprise one or more of probiotic bacteria in addition to the active agents from Bacillus velezensis (such as the Bacillus velezensis fermentate and/or the Bacillus velezensis metabolite).

The skin care composition can comprise additional compounds selected from the group consisting of preservatives, pH adjusters, anti-oxidants and chelators.

Preservatives include but are not limited to parabens, sodium benzoate, potassium sorbate, phenyl ethyl alcohol, Lauryl ethyl arginate (LAE) and any combination thereof.

pH adjusters include but are not limited to weak acids, strong acids, any compound that can adjust the pH, such as but not limiting to citric acid, or any combination thereof.

The skin care compositions or any effective amount of said skin care composition described herein can be used in formulations and skin care products.

As used herein “skin care products” refer to products comprising an effective amount of the skin care compositions described herein, including but not limiting to cosmetic products, aqueous solutions, emulsions, serums, jellies, patches, lotions, topical moisturizers, creams, pastes, balms, ointments, pomades, gels, liquids, sprays, foam, kits, or any one combinations thereof.

In one embodiment, the skin care product is formulated for topical administration to the skin/scalp.

In one embodiment, the composition is a skin care product comprising an effective amount of a skin care composition for use in the treatment of a scalp disorder, comprising an effective amount of a Bacillus velezensis fermentate, and/or metabolite thereof, wherein said composition reduces and/or treats said scalp disorder, and one or more dermatologically or skin care acceptable component.

In one embodiment, the skin care product is a lotion, a serum, a jelly, a cream, a gel, an emulsion, a mask, a patch, or a stick comprising one or more dermatologically or skin care acceptable components and at least about 1%, 2%, 3%, 4%, 5%, 6%, 7%, 8%, 9% up to 10% of the skin care formulation described herein on a weight basis relative to a total weight of said skin care formulation.

In one embodiment, the skin care product is a product comprising an effective amount of a Bacillus velezensis fermentate, and/or metabolite thereof, wherein the microorganism and/or the skin care composition comprising said microorganism is formulated in at least one form selected from the group consisting of a gel, an emulsion, a hydrogel, a loose or compact powder, a liquid suspension or solution, a spray solution, or any combination thereof.

The topical formulation for use in the present invention may be in any form suitable for application to the scalp or skin surface, such as a cream, lotion, sprays, solution, gel, ointment, paste, plaster, paint, bioadhesive, suspensions or the like, and/or may be prepared so as to contain liposomes, micelles, and/or microspheres. Such a formulation may be used in combination with an occlusive overlayer so that moisture evaporating from the body surface is maintained within the formulation upon application to the body surface and thereafter.

Topical formulations include those in which the active ingredient(s) is (are) dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy) ethanol, propylene glycol, propylene glycol monolaurate, glycofurol or glycerol), oils (e.g. a mineral oil such as a liquid paraffin, natural or synthetic triglycerides, or silicone oils such as dimethicone). Depending, inter alia, upon the nature of the formulation as well as its intended use and site of application, the dermatological vehicle employed may contain one or more components (for example, when the formulation is an aqueous gel, components in addition to water) selected from the following list: a solubilizing agent or solvent (e.g. a 3-cyclodextrin, such as hydroxypropyl β-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A skin care product includes a liquid lotion (true solution) comprising water as a solvent and watersoluble additives (solutes), such as but not limiting to an active, a fragrance, a color, a preservative, a pH adjuster, a chelating agent, or any one combination thereof.

A skin care product includes a dispersion such as an emulsion (such as, but not limited to the following: liquid in liquid [water in oil W/O, O/W, W/O/W], suspension [solid/liquid or liquid/solid], aerosol [liquid/gas or solid/gas], foam/mousse [gas/liquid or gas/emulsion, or gas/solid]). An example of an Oil in Water [O/W] emulsion includes, but is not limited to a combination of a water phase, an emulsifier, a fatty phase and an at least one additive. The water phase can comprise water, humectants and stabilizing agents [such as, but not limiting to, synthetic polymers, carbomers, natural polymers, xanthan gum, acacia gum, carragheenan, gellan, or any one combination thereof). Emulsifiers include, but are not limited to, anionic emulsifiers, cationic emulsifiers, non-ionic emulsifiers, amphoteric emulsifiers, silicone emulsifiers), auto emulsifying agents. Fatty phases (lipophilic ingredients) include, but are not limited to, waxes, butter, fatty esters, triglycerides, vegetal oil, mineral oil (parffinum), silicones, and thickeners/oil jellifying agents. Additives include, but are not limited to, preservative, fragrance (most often lipophilic), color, anti-oxidant, chelating agent, actives, pH adjuster (citric acid, lactic acid, AHA), neutralizers/strong basic agent like NaOH, Trimethylamine (for acrylic polymers to jellify) and powders.

A skin care product includes an aqueous gel comprising a water phase (including water, humectants, actives), a jellifying agent (such as but not limited to synthetic polymers, natural polymers, xanthan gum, acacia gum, carragheenan, gellan) and an additive (such as but not limited to fragrance, high HLB surfactant, color, actives, preservative system, pH adjuster, neutralizing agent, powders).

A skin care product includes a cleansing/surfactant system (such as but not limited to a shampoo, shower gel, micellar water) comprising a water phase (water, humectants), a surfactant, an additive (such as but not limited to fragrance, high HLB surfactant, color, actives, preservative system, pH adjuster, neutralizing agent, powders) and optionally a jellifying agent (such as but not limited to synthetic polymers, natural polymers, xanthan gum, acacia gum, carragheenan, gellan).

As described herein, a skin care product or formulation comprising an effective amount of a Bacillus velezensis fermentate, and/or metabolite thereof, as described herein, provides a skin care benefit selected from the group consisting of preventing a dandruff condition, reducing a dandruff condition, treatment of a dandruff condition, reducing the occurrence of Malassezia species (Malassezia spp.) and/or Staphylococcus species on the skin/scalp, improving the barrier function of the skin, skin moisturizing (protecting the skin/scalp against dehydration by maintaining, restoring and/or strengthening the moisturization of the skin) or any one combination thereof.

A dermatologically or skin care acceptable carrier may also be incorporated in the skin care product (formulation) of the present invention and may be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

The term “dermatologically acceptable” or “dermatologically acceptable carrier” or “skin care acceptable” or “skin care acceptable carrier” is used herein to refer to a compound or composition that may be incorporated into a dermatologically or skin care formulation without causing undesirable biological effects or unwanted interaction with other components of the formulation.

“Carriers” or “vehicles” as used herein refer to carrier materials suitable for incorporation in a topically applied composition. Carriers and vehicles useful herein include any such materials known in the art, which are nontoxic and do not interact with other components of the formulation in which it is contained in a deleterious manner.

The term “aqueous” refers to a formulation that contains water or that becomes water-containing following application to the skin or mucosal tissue.

Skin care products described herein may further comprise one or more dermatologically or skin care acceptable components known or otherwise effective for use skin care, provided that the optional components are physically and chemically compatible with the essential components described herein, or do not otherwise unduly impair product stability, aesthetics, or performance. Non-limiting examples of such optional components are disclosed in International Skin Care Ingredient Dictionary, Ninth Edition, 2002, and CTFA Skin Care Ingredient Handbook, Tenth Edition, 2004.

In one aspect, the dermatologically or skin care acceptable component is a dermatologically acceptable carrier comprising from about 10 wt. % to about 99.9 wt. %, alternatively from about 50 wt. % to about 95 wt. %, and alternatively from about 75 wt. % to about 95 wt. %, of a dermatologically acceptable carrier. Carriers suitable for use with the composition(s) may include, for example, those used in the formulation of mousses, tonics, gels, skin moisturizers and lotions. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheat germ oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one aspect, the carrier comprises water, fatty alcohols, volatile organic alcohols, and mixtures thereof. Other carriers can be formulated by those of ordinary skill in the art.

The skin care products described herein may further comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ S091 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers.

The dermatologically or skin care acceptable medium may contain a fatty substance in a proportion generally of from about 10 to about 90% by weight relative to the total weight of the product, where the fatty phase containing at least one liquid, solid or semi-solid fatty substance. The fatty substance includes, but is not limited to, oils, waxes, gums, and so-called pasty fatty substances. Alternatively, the products may be in the form of a stable dispersion such as a water-in-oil or oil-in-water emulsion. Additionally, the skin care products may contain one or more conventional skin care or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

The dermatologically acceptable carrier may be a moisturizer formulation containing at least one emulsifiers, at least one surfactant, or any combination thereof.

In one embodiment, the skin care product is a product comprising a first skin care composition and a second skin care composition, wherein the first skin care composition comprises an effective amount of a Bacillus velezensis fermentate, and/or metabolite thereof, wherein the second skin care composition comprises at least an effective amount of at least one second active agent (such as antidandruff active agents, skin conditioning agents, skin care active ingredient materials) for topical administration.

Skin care compositions and skin care products can further comprise skin care active ingredient materials including sun screen agents, moisturizers, humectants, benefiting agents skin, depositing agents such as surfactants, occlusive agents, moisture barriers, lubricants, emollients, anti-aging agents, antistatic agents, abrasive, antimicrobials, conditioners, exfoliants, fragrances, viscosifying agents, salts, lipids, phospholipids, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

Other ingredients that may be included in a skin care composition or skin care product include, without limitation, at least one active ingredient for the treatment or prevention of skin ailments, providing a skin care effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these, one or more natural moisturizing factors (such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example), glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

Any number of dermatologically acceptable materials commonly used in skin care products may also be incorporated into the present skin care products such as skin conditioning agents and skin colorants.

Skin conditioning agents as herein defined include, but are not limited to astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Skin conditioning agents are well known in the art, see for example Green et al. (WO01/07009), and are available commercially from various sources. Suitable examples of skin conditioning agents include, but are not limited to, lactobionic acid, gluconic acid, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils, xylitol, fucose, rhamnose, xylitol, betaine, and triglyceride esters. The skin conditioning agents may include polysalicylates, propylene glycol (CAS No. 57-55-6, Dow Chemical, Midland, MI), glycerin (CAS No. 56-81-5, Proctor & Gamble Co., Cincinnati, OH), glycolic acid (CAS No. 79-14-1, DuPont Co., Wilmington, DE), lactic acid (CAS No. 50-21-5, Alfa Aesar, Ward Hill, MA), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, DE). Polysalicylates may be prepared by the method described by White et al. in U.S. Pat. No. 4,855,483, incorporated herein by reference. Glucaric acid may be synthesized using the method described by Merbouh et al. (Carbohydr. Res. 336:75-78 (2001). The 3-hydroxyvaleric acid may be prepared as described by Bramucci in published international patent application number WO 02/012530.

Skin care compositions and skin care products can comprise skin care additives such as, but not limiting to, colorants/dyes, fragrances, actives, preservatives, pH adjusters, chelators, and anti-oxidants.

In one embodiment, the skin care product is a product comprising a first skin care composition and a second skin care composition, wherein the first skin care composition comprises an effective amount of a Bacillus velezensis fermentate, and/or metabolite thereof wherein the second skin care composition comprises at least an effective amount of at least one second active agent selected from antidandruff active agents, for topical administration. Examples of such antidandruff active agents include keratolytc agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones (e.g. ciclopirox), naturopathic agents such as Melaleuca sp. oil, Aloe vera, and probiotic microorganisms. (Indian J. Dermatol, 2010 April-June; 55(2): 130-134).

In one embodiment, the skin care product is a product comprising a first skin care composition and a second skin care composition, wherein the first skin care composition an effective amount of a Bacillus velezensis fermentate, and/or metabolite thereof; wherein the second skin care composition comprises at least an effective amount of at least one second active agent selected from antidandruff active agents, for topical administration, wherein the first skin care composition is formulated in at least one form selected from the group consisting of a gel, an emulsion, a hydrogel, a loose or compact powder, a liquid suspension or solution, or a spray solution.

Skin care compositions and skin care products described herein can also be part of a kit for providing one or more skin care benefits such as, but not limiting to, a kit for preventing or reducing a dandruff condition

In one aspect the kit is a kit comprising an effective amount of a Bacillus velezensis fermentate, and/or metabolite thereof for the treatment of a dandruff condition and written instructions for administration to the subject in need.

In one aspect the kit is a kit comprising a skin care product for the treatment of a dandruff condition of a subject in need, wherein said skin care product comprises an effective amount of a Bacillus velezensis fermentate, and/or metabolite thereof, and written instructions for administration said skin care product to the subject in need.

### Methods for Microbial Treatment of a Scalp Disorder

The skin care compositions and skin care products described herein can be used in methods for treating a scalp disorder.

Further provided herein are methods for treating a scalp disorder in a subject in need thereof, comprising administering an effective amount of a Bacillus velezensis fermentate, and/or metabolite thereof, to said subject.

In one aspect, the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

In one aspect, the Bacillus velezensis fermentate is administered topically.

In one aspect, the Bacillus velezensis fermentate is a fermentate of at least one Bacillus velezensis strain selected from the group consisting B. velezensis E04, B. velezensis H02, B. velezensis F03, and any one combination thereof.

In one embodiment, the method is a method for treating a scalp disorder in a subject in need thereof, comprising administering an effective amount a Bacillus velezensis fermentate, and/or metabolite thereof, to said subject.

In one embodiment, the method is a method for treating a scalp disorder in a subject in need thereof, comprising administering a composition comprising an effective amount a Bacillus velezensis fermentate, and/or metabolite thereof, wherein said composition reduces and/or treats said scalp disorder.

Methods for Treating and/or Reducing a Dandruff Condition.

The skin care compositions and skin care products described herein can be used in methods for treating a dandruff condition.

Further provided herein are methods for treating a dandruff condition in a subject in need thereof, comprising administering an effective amount of a Bacillus velezensis fermentate, and/or metabolite thereof, to said subject.

In one embodiment, the method is a method for treating and/or reducing a dandruff condition of the scalp in a subject in need thereof, comprising administering an effective amount of a Bacillus velezensis fermentate, and/or metabolite thereof, to said subject.

In some embodiments provided herein, the method comprises administering an effective amount of a Bacillus velezensis fermentate, and/or a Bacillus velezensis cell lysate, and/or a Bacillus velezensis fraction, and/or a Bacillus velezensis metabolite, for treating a scalp disorder, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one aspect, the Bacillus velezensis fermentate is administered topically.

In one aspect, the Bacillus velezensis fermentate is a fermentate of at least one Bacillus velezensis strain selected from the group consisting B. velezensis E04, B. velezensis H02, B. velezensis F03, and any one combination thereof.

In one embodiment, the method is a method for treating and/or reducing a dandruff condition of the scalp in a subject in need thereof, comprising topically administering a skin care product comprising a skin care composition described herein to said subject.

In one embodiment, the method is a method for treating and/or reducing a dandruff condition of the scalp in a subject in need thereof, comprising administering an effective amount of a Bacillus velezensis fermentate, and/or metabolite thereof, to said subject, wherein the Bacillus velezensis fermentate, and/or metabolite thereof, is formulated in a single composition, and wherein the composition is administered to the subjects' skin or scalp.

In one embodiment, the method is a method for treating and/or reducing a dandruff condition of the scalp in a subject in need thereof, comprising administering an effective amount of a Bacillus velezensis fermentate, and/or metabolite thereof, to said subject, wherein the Bacillus velezensis fermentate, and/or metabolite thereof, is formulated in a single composition, wherein the composition further comprises a compound selected from the group consisting of a skin care excipient, butyric acid, glucose, glycogen, magnesium ascorbyl phosphate, cetyl alcohol, dimethicone, isopropyl myristate, glycerol, propylene glycol, Quaternium-52, ethanol or any one combination thereof.

In one embodiment, the method is a method for treating and/or reducing a dandruff condition of the scalp in a subject in need thereof, comprising administering a skin care composition for use in the treatment of a dandruff condition, wherein said skin care composition comprises an effective amount of a Bacillus velezensis fermentate, and/or metabolite thereof, wherein said composition reduces and/or treats said dandruff condition.

In one embodiment, the method is a method for treating and/or reducing a dandruff condition of the scalp in a subject in need thereof, comprising administering a skin care product for use in the treatment of a dandruff condition to said subject, wherein said skin care product comprises a skin care composition comprising an effective amount of a Bacillus velezensis fermentate, and/or metabolite thereof, wherein said composition reduces and/or treats said dandruff condition.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 50 kDa, wherein said composition reduces and/or treats said scalp disorder.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 50 kDa, wherein said composition reduces growth of Malassezia species.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 30 kDa, wherein said composition reduces and/or treats said scalp disorder.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 30 kDa, wherein said composition reduces growth of Malassezia species.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 10 kDa, wherein said composition reduces and/or treats said scalp disorder.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 10 kDa, wherein said composition reduces growth of Malassezia species.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 3 kDa, wherein said composition reduces and/or treats said scalp disorder.

In one embodiment, the method is a method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 3 kDa, wherein said composition reduces growth of Malassezia species.

In one embodiment, the method is a method for treating and/or reducing a dandruff condition of the scalp in a subject in need thereof, comprising administering a skin care product for use in the treatment of a dandruff condition to said subject, wherein said skin care product comprises a skin care composition comprising an effective amount of a Bacillus velezensis fermentate, and/or metabolite thereof, wherein the Bacillus velezensis fermentate, and/or metabolite thereof, reduces the growth of Staphylococcus and/or Malassezia species.

In one embodiment, the method is a method for treating and/or reducing a dandruff condition of the scalp in a subject in need thereof, comprising administering a skin care product for use in the treatment of a dandruff condition to said subject, wherein said skin care product comprises a skin care composition comprising an effective amount of a Bacillus velezensis fermentate, and/or metabolite thereof, wherein the Bacillus velezensis fermentate, and/or metabolite thereof removes biofilm of Malassezia species and/or Staphylococcus species.

In one aspect, the method is a method for treating and/or reducing a dandruff condition of the scalp in a subject in need thereof, comprising topically administering a skin care product comprising a skin care composition described herein.

In one aspect, the method is a method for treating and/or reducing a dandruff condition of the scalp in a subject in need thereof, comprising topically administering a skin care product comprising a skin care composition described herein, wherein the skin care product is a selected from the group consisting of a lotion, a serum, a jelly, a cream, a gel, an emulsion, a solid cosmetic, a mask, a patch, and a stick comprising at least 1%, 2%, 3%, 4% up to 5% of said skin care composition on a weight basis relative to a total weight of said skin care product.

In one embodiment, the method is a method for treating and/or reducing a dandruff condition of the scalp in a subject in need thereof, comprising administering an effective amount of a Bacillus velezensis fermentate, and/or metabolite thereof, to said subject.

### General Definitions

The disclosures of all cited patent and non-patent literature are incorporated herein by reference in their entirety.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein “administer” or “administering” is meant the action of introducing one or more microorganism (microbial strain), skin care composition(s), skin care formulation(s) and/or skin care product(s) to a subject in need for treatment of a scalp disorder.

Administering one or more microorganism (microbial strain), skin care composition(s), skin care formulation(s) and/or skin care product(s) to a subject includes applying or introducing one or more microorganism (microbial strain), skin care composition(s), skin care formulation(s) and/or skin care product(s) to a scalp, a skin surface, and to in-vitro or in-vivo skin cells.

As used herein, the term “biological contaminants” refers to one or more unwanted and/or pathogenic biological entities including, but not limited to, microorganisms, spores, viruses, prions, and mixtures thereof.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “embodiment” or “disclosure” is not meant to be limiting, but applies generally to any of the embodiments defined in the claims or described herein. These terms are used interchangeably herein.

As used herein, the term “excipient” refers to inactive substance used as a carrier for active ingredients, in a formulation. The excipient may be used to stabilize the active ingredient in a formulation, such as the storage stability of the active ingredient. Excipients are also sometimes used to bulk up formulations that contain active ingredients. An “active ingredient” includes a skin care benefit agent as described herein.

As used herein, the term “effective amount” refers to the amount sufficient to obtain the desired effect. A desired effect includes the prevention, reduction and/or treatment of a scalp disorder, such as the prevention, reduction and or treatment of dandruff condition.

As used herein, “prevent,” “preventing,” “prevention” and grammatical variations thereof refers to a method of partially or completely delaying or precluding the onset or recurrence of a disorder or condition (such as a scalp disorder) and/or one or more of its attendant symptoms or barring a subject from acquiring or reacquiring a disorder or condition or reducing a subject's risk of acquiring or reacquiring a disorder or condition or one or more of its attendant symptoms.

As used herein, the term “reducing”, “reduces” and grammatical variations thereof in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

The terms “percent by weight”, “weight percentage (wt. %)” and “weight-weight percentage (% w/w)” are used interchangeably herein. Percent by weight refers to the percentage of a material on a mass basis as it is comprised in a composition, mixture, solution or product.

The term “16S rRNA” or “16S ribosomal RNA” means the rRNA constituting the small subunit of prokaryotic ribosomes. In bacteria, this sequence can be used to identify and characterize operational taxonomic units.

The term “ITS” or “Internal Transcribed Spacers” are regions within the ribosomal transcript that are excised and degraded during maturation. Their sequences can be used for phylogenetic analysis and/or identification of fungi or yeast.

The terms moisturizer, a lotion or a body lotion refer to a low to medium-viscosity emulsion of oil and water, most often oil-in-water but possibly water-in-oil with the primary benefit in a skin care application to hydrate the skin or to reduce its water loss. Nearly all moisturizers contain a combination of emollients, occlusives, and humectants. Emollients, which are mainly lipids and oils, hydrate and improve the appearance of the skin. A wide variety of suitable emollients is known and maybe used herein (International Skin Care Ingredient Dictionary and Handbook, eds. Wenninger and McEwen, pp. 1656-61, 1626, and 1654-55 (The Skin care, Toiletry, and Fragrance Assoc., Washington, D.C., 7th Edition, 1997) (referred to as “ICI Handbook”) contains numerous examples of suitable materials). Occlusives such as petrolatum, lanolin and bee wax reduce transepidermal water loss by creating hydrophobic barrier over the skin. Humectants such as glycerol and urea able to attract water from the external environment and enhance water absorption from the dermis into the epidermis. In addition, the moisturizer formulations may contain emulsifiers to maintain stability of emulsions, and use thickeners to achieve desired viscosity and skin feel. A wide variety of other ingredients such as fragrances, dyes, preservatives, therapeutic agents, proteins and stabilizing agents are commonly added for other consumer preferred attributes.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

A microbial “strain” as used herein refers to a microorganism (such as a bacterium or fungus) which remains genetically unchanged when grown or multiplied. The multiplicity of identical microbes is included.

As used herein, the term a “biologically pure strain” means a strain containing no other microbial strains in quantities sufficient to interfere with replication of the strain or to be detectable by normal techniques. “Isolated” when used in connection with the organisms and cultures described herein includes not only a biologically pure strain, but also any culture of organisms which is grown or maintained other than as it is found in nature.

In one aspect the skin cells described herein are mammalian skin cells, such as human or animal skin cells.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Non-limiting examples of compositions and methods disclosed herein include:
1. A skin care composition for use in the treatment of a scalp disorder, comprising an effective amount of a Bacillus velezensis fermentate, and/or metabolite thereof, wherein said composition reduces and/or treats said scalp disorder. 1b. The skin care composition of embodiment 1, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
2. The skin care composition of embodiment 1, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.
3. The skin care composition of embodiment 1, further comprising one or more antidandruff active agents.
4. The skin care composition of embodiment 1, wherein the composition reduces the growth of Staphylococcus species and/or Malassezia species.
5. The skin care composition of embodiment 4, wherein the composition reduces the growth of Staphylococcus species selected from the group consisting Staphylococcus aureus and Staphylococcus epidermidis.
6. The skin care composition of embodiment 1, wherein the composition removes biofilm of Malassezia species and/or Staphylococcus species.
7. The skin care composition of embodiment 6, wherein the composition removes biofilm of Malassezia species and/or Staphylococcus species selected from the group consisting of Malassezia globosa and Staphylococcus aureus.
7b. The skin care composition of embodiment 4, wherein the composition reduces the growth of Malassezia species selected from the group consisting of Malassezia globosa, Malassezia restricta, Malassezia sympodialis, Malassezia furfur, Malassezia phylotype 5, other uncharacterized Malassezia species, and any combination thereof.
8. The skin care composition of embodiment 1, further comprising additional compound selected from the group consisting of an excipient, a preservative, a pH adjuster.
8b. A skin care composition for use in the treatment of a scalp disorder, comprising an effective amount of a Bacillus velezensis fermentate, wherein said Bacillus velezensis fermentate comprises one or more bioactive with a molecular weight of less than 50 kDa, wherein said composition reduces and/or treats said scalp disorder.
8c. A skin care composition for use in the treatment of a scalp disorder, comprising an effective amount of a Bacillus velezensis fermentate, wherein said Bacillus velezensis fermentate comprises one or more bioactive with a molecular weight of less than 50 kDa, wherein said composition reduces growth of Malassezia species.
8d. A skin care composition for use in the treatment of a scalp disorder, comprising an effective amount of a Bacillus velezensis fermentate, wherein said Bacillus velezensis fermentate comprises one or more bioactive with a molecular weight of less than 30 kDa, wherein said composition reduces and/or treats said scalp disorder.
8e. A skin care composition for use in the treatment of a scalp disorder, comprising an effective amount of a Bacillus velezensis fermentate, wherein said Bacillus velezensis fermentate comprises one or more bioactive with a molecular weight of less than 30 kDa, wherein said composition reduces growth of Malassezia species.
8f. A skin care composition for use in the treatment of a scalp disorder, comprising an effective amount of a Bacillus velezensis fermentate, wherein said Bacillus velezensis fermentate comprises one or more bioactive with a molecular weight of less than 10 kDa, wherein said composition reduces and/or treats said scalp disorder.
8g. A skin care composition for use in the treatment of a scalp disorder, comprising an effective amount of a Bacillus velezensis fermentate, wherein said Bacillus velezensis fermentate comprises one or more bioactive with a molecular weight of less than 10 kDa, wherein said composition reduces growth of Malassezia species.
8h. A skin care composition for use in the treatment of a scalp disorder, comprising an effective amount of a Bacillus velezensis fermentate, wherein said Bacillus velezensis fermentate comprises one or more bioactive with a molecular weight of less than 3 kDa, wherein said composition reduces and/or treats said scalp disorder.
8i. A skin care composition for use in the treatment of a scalp disorder, comprising an effective amount of a Bacillus velezensis fermentate, wherein said Bacillus velezensis fermentate comprises one or more bioactive with a molecular weight of less than 3 kDa, wherein said composition reduces growth of Malassezia species.
8j. The skin care composition of any preceding embodiment, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
9. Use of an effective amount of the skin care composition of any preceding embodiment in a skin care product.
10. A skin care product comprising an effective amount of the skin care composition of any preceding embodiment and one or more dermatologically or skin care acceptable component.
11. The skin care product of embodiment 10, wherein said effective amount of the skin care composition is at least about 1%, 2%, 3%, 4% up to 5% on a weight basis relative to a total weight of said skin care product.
12. The skin care product of embodiment 10, wherein the product is formulated for topical administration.
12b. A skin care product comprising a first skin care composition and a second skin care composition, wherein the first skin care composition comprises an effective amount of a first active agent consisting of Bacillus velezensis fermentate, and/or metabolite thereof; wherein the second skin care composition comprises at least an effective amount of at least one second active agent selected from antidandruff active agents for topical administration.
12c. The skin care product of embodiment 12b, wherein the first skin care composition is formulated in at least one form selected from the group consisting of a gel, an emulsion, a hydrogel, a loose or compact powder, a liquid suspension or solution, or a spray solution.
12d. The skin care product of embodiment 12b, wherein the at least one second composition comprises at least one member selected from the group consisting of a hair lotion, a shampoo, a hair conditioner, a detangler, a hair cream or gel, a styling lacquer, a hairsetting lotion, a treating lotion, a dye composition, a hair-restructuring lotion, a permanent-waving composition, a lotion or gel for combating hair loss, an antiparasitic shampoo or a medicated shampoo, and a scalp care product.
12e. The skin care product of embodiments 12, 12b, 12c or 12d, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
13. A method for treating a scalp disorder in a subject in need thereof, comprising administering an effective amount of a Bacillus velezensis fermentate, and/or metabolite thereof, to said subject.
14. The method according to embodiment 13, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.
15. The method according to embodiment 13, wherein the fermentate is administered topically.
16. A method for treating and/or reducing a dandruff condition of the scalp in a subject in need thereof, comprising administering an effective amount of a Bacillus velezensis fermentate, and/or metabolite thereof, to said subject.
16b. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 50 kDa, wherein said composition reduces and/or treats said scalp disorder.
16c. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 50 kDa, wherein said composition reduces growth of Malassezia species.
16d. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 30 kDa, wherein said composition reduces and/or treats said scalp disorder.
16e. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 30 kDa, wherein said composition reduces growth of Malassezia species.
16f. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 10 kDa, wherein said composition reduces and/or treats said scalp disorder.
16g. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 10 kDa, wherein said composition reduces growth of Malassezia species.
16h. A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 3 kDa, wherein said composition reduces and/or treats said scalp disorder.
16i A method for treating and/or reducing a dandruff condition of the scalp in a subject in need thereof, comprising administering a composition comprising an effective amount of a Bacillus velezensis fermentate to said subject, wherein said Bacillus velezensis fermentate comprises one or more bioactive with a molecular weight of less than 3 kDa, wherein said composition reduces growth of Malassezia species.
17. The method according to embodiment 16-16i, wherein the fermentate, and/or metabolite thereof, is administered topically.
18. The method of embodiment 16-16i, wherein the fermentate, and/or metabolite, is formulated in a single composition.
19. The method of embodiment 16-16i, wherein the composition is administered to the subject's skin or scalp.
20. The method of embodiment 16, wherein the fermentate, and/or metabolite thereof, reduces the growth of Staphylococcus species and/or Malassezia species.
21. The method of claim 20, wherein the fermentate, and/or metabolite thereof, reduces the growth of Malassezia species and/or Staphylococcus species selected from the group consisting of Staphylococcus aureus, Staphylococcus epidermidis, Staphylococcus haemolyticus, Staphylococcus capitis, other uncharacterized Staphylococcus species, Malassezia globosa, Malassezia restricta, Malassezia sympodialis, Malassezia furfur, Malassezia phylotype 5, other uncharacterized Malassezia species, and any combination thereof.
22. The method of embodiment 16, wherein the fermentate, and/or metabolite thereof, removes biofilm of Malassezia species and/or Staphylococcus species.
22b. The method of embodiment 16, wherein the fermentate, and/or metabolite thereof, removes biofilm of Malassezia globosa and Staphylococcus aureus.
23. The method of any one of embodiments 13-22, wherein the effective amount of a Bacillus velezensis fermentate, and/or metabolite thereof, is administered as a skin care composition or a skin care product.
23b. The method of embodiments 13-23, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
24. A method for treating and/or reducing a dandruff condition of the scalp in a subject in need thereof, comprising topically administering a skin care product comprising a skin care composition of any one of embodiment 1-8 to said subject.
25. The method of embodiment 24, wherein the skin care product is a selected from the group consisting of a lotion, a serum, a jelly, a cream, a gel, an emulsion, a solid cosmetic, a mask, a patch, and a stick comprising at least 1%, 2%, 3%, 4% up to 5% of said skin care composition on a weight basis relative to a total weight of said skin care product.
26. A method for treating and/or reducing a dandruff condition of the scalp in a subject in need thereof, comprising administering an effective amount of a first cosmetic active agent and an effective amount of at least one second cosmetic active agent, wherein the first cosmetic active agent consist of a f Bacillus velezensis fermentate, and/or metabolite thereof.
27. The method of embodiment 26, wherein the first and second cosmetic active are administered to the subject's skin or scalp.
28. The method of embodiment 26, wherein the first cosmetic active agent and the at least one second cosmetic active agent are formulated in a single composition.
29. The method of embodiment 26, wherein the first cosmetic active agent and the at least one second cosmetic active agent are formulated in separate compositions.
30. The method of embodiment 26, wherein the at least one second cosmetic active agent comprises at least one antidandruff active agent.
31. The of embodiment 30, wherein the at least one second cosmetic active agent comprises at least one member selected from the group consisting of zinc pyridinethione, salicylic acid, selenium disulfide, mixed oil of thyme and catnip, octopirox, a probiotic microorganism, and a microorganism.
32. The method of embodiment 31, wherein the at least one second cosmetic active agent comprises at least one member selected from the group consisting of keratolytc agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones such as (e.g. ciclopirox), naturopathic agents such as Melaleuca sp. oil, Aloe vera, and a probiotic microorganism.
33. The method of embodiment 32, wherein the dandruff condition of the scalp comprises dandruff in combination with: dryness of the scalp, hyperseborrhoea of the scalp, an imbalanced ecoflora, pruritus, inflammation of the scalp, or an imbalanced barrier function of the scalp.
34. A method for preparing a cosmetic composition or dermatological composition for treating a dandruff condition, comprising combining an effective amount of a Bacillus velezensis fermentate, and/or metabolite thereof, with at least one cosmetic or dermatological excipient.
35. A skin care composition for use in the treatment of a scalp disorder, comprising a fermentate of Bacillus velezensis cells, wherein said composition reduces and/or treats said scalp disorder.
36. The skin care composition of embodiment 35, wherein all or substantially all of the Bacillus velezensis cells have been removed from the fermentate.
37. The composition of embodiment 36, wherein the fermentate is a culture supernatant.
38. The composition of embodiment 36, further comprising one or more dermatologically or skin care acceptable component.
38b. The skin care product of embodiments 35-38, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
39. A skin care product comprising the skin care composition of any one of embodiments 37-38.
40. Fermentate of Bacillus velezensis for use in treatment of a scalp disorder.
41. Fermentate of Bacillus velezensis for use according to embodiment 40, wherein all or substantially all of the Bacillus velezensis cells have been removed from the fermentate.
42. Fermentate of Bacillus velezensis for use according to embodiment 40, wherein the fermentate is a culture supernatant.
42b. The fermentate of embodiments 40-42, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
43. A skin care product comprising the fermentate of any one of embodiments 40-42b.
44. A skin care composition for use in the treatment of a scalp disorder, comprising a metabolite of Bacillus velezensis cells, wherein said metabolite is derived from the metabolism of a Bacillus velezensis microorganism and also having efficacy in the treatment of said scalp disorder, wherein said composition reduces and/or treats said scalp disorder.
45. The skin care composition of embodiment 39, further comprising one or more dermatologically or skin care acceptable component.
46. A skin care product comprising the skin care composition of any one of embodiments 43-45.
47. A metabolite of Bacillus velezensis for use in treatment of a scalp disorder, wherein said metabolite is derived from the metabolism of a Bacillus velezensis microorganism and also having efficacy in the treatment of said scalp disorder.
47b. The metabolite embodiments 44 or 47, wherein the Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “conc.” means concentration, “Trt” means treatment.

### Example 1

Isolation of Microbes from Soil Rhizosphere

A collection of microbes from rhizosphere soil of corn fields were used to generate cell free supernatant (fermentate). Growth media used for the isolation campaign include R2A, tryptic soy agar (TSA), and nutrient agar (NA). R2A medium consists of casein acid hydrolysate (0.5 g/L), dextrose (0.5 g/L), dipotassium phosphate (0.3 g/L), magnesium sulfate (0.024 g/L), protease peptone (0.5 g/L), sodium pyruvate (0.3 g/L), soluble starch (0.5 g/L), and yeast extract (0.5 g/L). Each liter of tryptic soy agar contains 17.0 g tryptone, 3 g soytone, 2.5 g of glucose, 5 g of sodium chloride, 2.5 g of dipotassium phosphate, and 15 g of agar. NA medium has meat extract, 1 g/L, peptone, 5 g/L, sodium chloride, 5 g/L, yeast extract, 2 g/L and agar, 15 g/L.

Plant roots were excavated by piercing the soil with a shovel, and root-balls were cut to discard above ground plant mass. Bulk soil was removed by manually shaking. After shaking, soil closely associated with the root was collected in a wash pan with sterile water. Soil samples were suspended in sterile water. Serial dilutions up to 10⁴ were plated to R2A, TSA, and NA media for isolation of a variety of microbes. Agar plates were incubated at 28° C. for 24-72 hours. Colonies were picked, grown in liquid medium, and stored at −80° C. in 25% glycerol. The phylogenetic identity of isolates was determined by sequencing the 16S rRNA region with primer set 8F (5′-AGA GTT TGA TYM TCC TGG CTC-3, SEQ ID NO: 1) and 1492R (5′-CGG TTA CCT TGT TAC GAC TT-3, SEQ ID NO: 2).

### Example 2

Growth Inhibition Assays with Isolate Fermentate for Bacterial Targets

To generate cell free supernatants (also referred to as fermentate) from individual isolates described in Example 1, strains were revived from frozen stocks and grown in 1 mL TSB medium in deep-well microtiter plates. To prepare filtrate for microtiter plate assays, the candidate strains were sub-cultured by inoculating 25 μl of revived culture into 1100 μl fresh TSB in 96-deepwell microtiter plates and grown for three days at 28° C. in a chamber with 75% RH with shaking at 350 rpm. To collect fermentate, cell culture was centrifuged at >6,100 rcf at 4° C. Approximately 700 μl of the supernatant was removed and filtered through a 0.2 μm filter with a 96-well filter plate.

As described herein, Staphylococcus species are associated dandruff based on microbiome analysis of healthy and dandruff populations. For the growth inhibition assays, Staphylococcus aureus (S. aureus) ATCC 6538 and Staphylococcus epidermidis (S. epidermidis) ATCC 49461 were chosen as the targets.

Inhibition assay with fermentate of different soil isolates was carried out in a 384-well format. A fresh overnight shake flask cultures in TSB were diluted to equivalent McFarland turbidity standard 1.0 in 1×PBS (Phosphate-buffered saline). To prepare the starter culture stock used in the 384-well inhibition assay, this culture was then diluted again to 1:500 with a diluted TSB media (50% TSB+50% PBS (1× phosphate buffered saline solution). The 384-well inhibition assay was performed by adding 80 μl of the starter culture to each well with 20 μl of test fermentate.

The fermentate from each isolate was evaluated for inhibition in replicate wells (n=4) within each 384-well plate. Sterile TSB medium was used as a negative control. The 384-well assay plates were incubated at 32° C. for 18 hours, and growth in each well was measured by absorbance of OD (600 nm) using a plate spectrophotometer.

Fermentate from three strains of Bacillus velezensis, namely Bacillus velezensis E04 deposited at Westerdijk Fungal Biodiversity Institute (WFDB) under number CBS147469, Bacillus velezensis H02 deposited at Westerdijk Fungal Biodiversity Institute (WFDB) under number CBS 147473, and Bacillus velezensis F03 deposited at Westerdijk Fungal Biodiversity Institute (WFDB) under number CBS147471 (Table 1) were identified to have efficacy based on inhibition assays. As shown in Table 2, fermentate from these three strains had strong activity in inhibiting the growth of S. aureus and S. epidermidis species. Both two Staphylococcus species have been found to be associated with dandruff.

TABLE 1
List of Bacillus velezensis strains

| | DNA sequence | RNA sequence |
|---|---|---|
| Strains | of 16S rRNA | of 16S rRNA |
| B. velezensis E04 | SEQ ID NO: 3 | SEQ ID NO: 6 |
| B. velezensis H02 | SEQ ID NO: 4 | SEQ ID NO: 7 |
| B. velezensis F03 | SEQ ID NO: 5 | SEQ ID NO: 8 |

TABLE 2
Percent (%) growth inhibition of Bacillus velezensis strains for
targets S. aureus ATCC 6538 and S. epidermidis ATCC 49461

| | % inhibition | % Inhibition |
|---|---|---|
| | S. aureus | S. epidermis |
| B. velezensis E04 | 99.3 | 76.3 |
| B. velezensis H02 | 100 | 77.7 |
| B. velezensis F03 | 92.4 | 74.9 |

### Example 3

Removal of Biofilm of Malassezia Species by Fermentate from Bacillus velezensis Strains

In this example, yeast Malassezia globosa (M. globosa) ATCC MYA-4612 was used for biofilm assay to evaluate the fermentate from three strains of Bacillus velezensis, namely Bacillus velezensis E04 deposited at Westerdijk Fungal Biodiversity Institute (WFDB) under number CBS147469, Bacillus velezensis H02 deposited at Westerdijk Fungal Biodiversity Institute (WFDB) under number CBS 147473, and Bacillus velezensis F03 deposited at Westerdijk Fungal Biodiversity Institute (WFDB) under number CBS147471. The yeast strain was routinely grown in MLN media plus palm oil at 32° C. in a rotary shaker with a speed of 100 rpm. MLN medium consists of 10 g/L bacteriological peptone, 2 g/L yeast extract, 8 g/L desiccated ox bile, 10 ml/L glycerol, 0.5 g/L glycerol monostearate, and 5 ml/L Tween-60. Palm oil was supplemented at a concentration of 20 ml/L. After 3 days of incubation, 10 μl fresh culture was inoculated into wells of a polystyrene 96 well plate with 90 μl of MLN media using palm oil as the carbon source. The plate was incubated at 32° C. without shaking for 48 hours to allow growth of M. globosa as sessile biofilm cells attached to the wall of wells in the microtiter plate.

To generate the fermentate, different strains were grown in TSB medium at 28° C. for 3 days with 1 ml medium in a deep well blocks in a shaking incubator with 75% RH and a shaking speed of 350 rpm. The cells were collected by centrifugation at a speed of 4000 rpm with a table top centrifuge and the supernatant was filtered with a sterile 96-well 0.2 μm membrane filter unit before use (thus generating the fermentate). To generate larger volume of fermentate, selected strains were grown in 25 ml TSB in a 100 ml shake flask. The flask was placed at 32° C. for 2 days in a shaking incubator with a speed 200 rpm.

In the biofilm assay with a microtiter plate, the growth medium and the unattached cells were removed, and wells were washed once with 1× phosphate buffered saline solution (PBS) after the biofilm formation. PBS is a pH-adjusted blend of ultrapure-grade phosphate buffers and saline solutions which, when diluted to a 1× working concentration, contains 137 mM NaCl, 2.7 mM KCl, 8 mM Na2HPO4, and 2 mM KH2PO4. After washed, 150 μl supernatant of bacterial strains was added to evaluate their efficacy for biofilm removal. 150 μl TSB was also added as a control. The biofilm plate was incubated at 32° C. for 15 min without shaking to carry out the biofilm removal reaction. After incubation, the supernatant or control medium was removed. The amount of biofilm remaining in the wells were quantified by staining. The staining of biofilm was carried out by adding 150 μl of 0.1% crystal violet dissolved in water. The plate was incubated for 3 min at room temperature. After staining, 150 μl of 1×PBS was added to each well to remove the unbound dye. This process was repeated 1 more time. After wash, 150 μl of 70% ethanol was added to each well and the plate incubated for 5 min at room temperature to release the dye. The intensity of the dye was measured at 570 nm with a microtiter plate reader. The OD reading was used to quantify the amount of biofilm remained after each treatment. The wells treated with TSB medium were used as a reference to calculate the percentage of biofilm removal.

After screening for Malassezia biofilm removal, fermentate from soil isolates described in Example 1, the same three B. velezensis listed in Table 1, were also found to have the activity to remove Malassezia biofilm. Results of their activity for Malassezia biofilm removal using fermentate generated from flasks is shown in Table 3. Addition of fermentate from these strains led to a biofilm reduction by 77 to 80% as compared to the TSB control. This experiment demonstrated the potential usefulness of supernatant from Bacillus velezensis strains to control Malassezia biofilm.

TABLE 3
Removal of Malassezia biofilm by fermentate
of Bacillus velezensis strains.

| | OD | % biofilm |
|---|---|---|
| | (570 nm) | removal |
| TSB medium | 2.56 | 0.0 |
| B. velezensis E04 | 0.51 | 80.2 |
| B. velezensis H02 | 0.57 | 77.9 |
| B. velezensis F03 | 0.56 | 78.2 |

### Example 4

Removal of Staphylococcus aureus Biofilm by Bacillus velezensis Fermentate

Staphylococcus aureus (S. aureus) ATCC 6538 was used for biofilm experiment to evaluate the efficacy of B. velezensis supernatant (fermentate) from three strains of Bacillus velezensis, namely Bacillus velezensis E04 deposited at Westerdijk Fungal Biodiversity Institute (WFDB) under number CBS147469, Bacillus velezensis H02 deposited at Westerdijk Fungal Biodiversity Institute (WFDB) under number CBS 147473, and Bacillus velezensis F03 deposited at Westerdijk Fungal Biodiversity Institute (WFDB) under number CBS147471. Tryptic soy broth medium (TSB) was routinely used for growth of S. aureus. TSB (per liter) consists of 17.0 g tryptone, 3.0 g soytone, 2.5 g glucose, 5.0 sodium chloride, 2.5 g dipotassium phosphate at a pH of 7.3. Biofilm formation medium (BFM) consists of 50% BHI (brain heart infusion) and 50% YPD (yeast peptone dextrose).

B. velezensis strains were grown in TSB medium at 28° C. for 3 days with 1 ml medium in a deep well blocks for higher throughput or at 32° C. for two days with 25 ml medium in a flask to obtain larger volume. With a deep well blocks, the cultures were incubated in a shaker with a speed of 350 rpm. With flasks, the cultures were incubated in a shaker with a speed of 200 rpm. Supernatant (fermentate) was generated from cultures after 48 hours of growth in a rotary shaker with a speed of 200 rpm. The cells were collected by centrifugation at a speed of 4000 rpm with a table top centrifuge and the supernatant was filtered with a 0.2 μm membrane filter before use.

For biofilm assay, a 96-well sterile polystyrene microtiter plate was used. Ten microliters of a two-hour culture of S. aureus from a fresh plate was transferred to each well containing 90 μl of biofilm formation medium (BFM). The plate was incubated at 37° C. to allow biofilm formation on the wall of each well. After 24 hours of incubation, the growth medium was removed and 100 μl of 1×PBS was added to each well to gently wash and removed the unattached cells. To test the B. velezensis fermentate for S. aureus biofilm removal, 100 μl of supernatant of was added to each well. As controls, 100 μl of TSB was used. The plate was incubated at 37° C. for 20 min for the biofilm removal reaction. After the incubation, the solution in each well was removed and the wells were washed with 1×PBS. After emptying PBS solution, the attached biofilm was stained for 3 min at room temperature with 100 μl of 0.1% crystal violet dye dissolved in water. The unbound dye was removed, and each well was washed two times with 1×PBS. After emptying PBS solution, 100 μl of 70% ethanol was added to suspend the dye bound to the biofilm. The dye intensity was measured at 570 nm with a spectrophotometer. The OD was used to indicate the amount of biofilm remained after treatment.

The result of biofilm removal was shown in Table 4. As compared to the TSB control, the biofilm removal activity for S. aureus was 70% or higher with fermentate from these three strains.

TABLE 4
Removal of S. aureus biofilm by fermentate
of B. velezensis strains.

| | % biofilm |
|---|---|
| | removal |
| TSB medium | 0 |
| B. velezensis E04 | 76.9 |
| B. velezensis H02 | 70.7 |
| B. velezensis F03 | 70.8 |

This result in combination with the results from Example 2 and 3 demonstrated the unexpected feature of Bacillus velezensis strains in the removal of biofilms formed by two important species Staphylococcus and Malassezia that are associated with dandruff and scalp disorders.

### Example 5

Growth Inhibition of Malassezia Species by B. velezensis Fermentates.

The ability of B. velezensis fermentate to inhibit the growth of Malassezia species was evaluated. B. velezensis E04 strain was grown in SUM (Soyptone Urea Micronutrient) medium. Each liter of SUM medium (1.0× strength) consists of 10 g soytone, 75 g glucose, 3 ml of dipotassium phosphate (1 M), 3.6 g urea, and 10 ml of micronutrient stock. Each liter of the micronutrient stock contained 1.47g Sodium Citrate-2H₂O, 1.47 CaCl₂·2 H₂O, 0.4 FeSO4·7 H₂O, 0.1 g MnSO4·H₂O, 0.1 g ZnSO4·H₂O, 0.05 g CuCl₂·2 H₂O, 0.1 g CoCl₂·6 H₂O, and 0.1 g Na2MoO4·2 H₂O. The SUM medium was adjusted to pH 7.3. Generation of B. velezensis fermentate was carried out in 125 ml flat bottom shake flasks with 25 ml medium. The flasks were placed in a platform shaker with a constant speed of 200 rpm at 32° C. The overnight fresh seed culture was used as inoculum and the starting OD was 0.125 at 600 nm. After growth for 48 hours, the supernatant (fermentate) was obtained by centrifugation and filter-sterilization.

To determine the antifungal activity of fermentate of B. velezensis, growth inhibition assay was performed with Malassezia furfur (M. furfur) as the target. M. furfur ATCC 14521 was cultured by inoculating 1:100 from frozen stock into mDixon media containing 500 μg/mL chloramphenicol and incubated at 32° C. in an orbital shaker at 250 rpm for 2 days before the antifungal susceptibility test. Each liter of mDixon medium contains 36 g malt extract, 20 g desiccated oxbile, 10 ml Tween-40, 6 g peptone, 2 ml glycerol, and 2 ml oleic acid. The medium pH was adjusted to 6. The M. furfur cells were suspended in 1% Tween-80 in order to obtain an optical density equivalent to turbidity standard McFarland 4.0. This standardized cell culture was diluted once more at 1:10 ratio in Sabouraud Dextrose broth containing 500 μg/mL chloramphenicol with Tween-80 added to 1%. Sabouraud Dextrose broth consists of dextrose (40 g/L) and peptone (10 g/L) with pH adjusted to 5.6.

In the growth inhibition assay, the final volume of reaction was 600 μl and the reaction was performed in 96-deepwell plates (Corning Costar 3960). First, a dilution series of fermentate was generated by diluting the fermentate by 2, 4, and 8 fold in PBS (phosphate-buffer saline). PBS consists of 8 g of NaCl, 0.2 g KCl, 1.42 g Na2HPO4, and 0.24 g of KH2PO4 per liter. 150 μL of the undiluted and diluted fermentate was dispensed in each well of the 96-deepwell plates. 450 μL of the standardized culture of M. furfur in SabDex+Tween-80 was then added to each well of the 96-deepwell plate containing different concentrations of fermentate. The final concentration of fermentate in each reaction by volume was 25% for the undiluted fermentate, and 12.5%, 6.25%, and 3.125%, respectively, for the diluted fermentate. The assay plates were incubated in an orbital shaker at 35° C. with 75% relative humidity with shaking speed of 375 rpm at 25 mm orbital throw. After 48 hours, optical density measurement at the wavelength of 600 nm by a microplate reader. The results were reported as % inhibition (Table 5). The percent of Inhibition was calculated as =100*(1−(Observed−Min)/(Max−Min)), where Observed=Measure OD (average of test replicates), Max=Average OD of Negative controls with PBS (no fermentate), and Min=OD where no growth was observed (without M. furfur).

TABLE 5
Percent (%) growth inhibition of Malassezia fungal
targets by Bacillus velezensis fermentates

| | % inhibition |
|---|---|
| | M. furfur |
| B. velezensis E04 (25.0% fermentate) | 97.9 to 100% |
| B. velezensis E04 (12.5% fermentate) | 99.1 to 100% |
| B. velezensis E04 (6.25% fermentate) | 99.5 to 100% |
| B. velezensis E04 (3.125% fermentate) | 99.8 to1 00% |

As shown in Table 5, Bacillus velezensis fermentates were able to provide strong growth inhibition of Malassezia species. No significant difference in effect of Bacillus velezensis fermentate on Malassezia inhibition (ranging from 99.1 to 100%) was observed when B. velezensis fermentate originated from cultures that were grown in SUM 1.0× strength., SUM 0.5× strength, or SUM 0.25× strength.

### Example 6

Fractionation of Fermentate of Bacillus velezensis

To analyze the molecular weights of bioactives with antifungal activity, fermentate of B. velezensis was obtained from cells grown on 0.5×SUM medium as described in Example 5. Fractionation of fermentate was carried out with EMD Amicon ultra-0.5 centrifugal filter units with a molecular cut-off of 10, 30, and 50 kilodalton (kDa). The flow through was used in the antifungal assay with M. furfur as the target as described in Example 5. Table 6 showed the assay result with 3.125% fermentate. Up to 99% of the activity was found in the flow through of 50, 30, and 10 KDa, suggesting a majority of the bioactives responsible for antifungal activity had a molecular weight of 10 kDa or less.

TABLE 6
Antifungal activity of different fractions
of B. velezensis fermentate.

| | % Inhibition |
|---|---|
| Unfractionated | 100.0 |
| 50 kDa flow-through | 99.8 |
| 30 kDa flow-through | 99.7 |
| 10 kDa flow-through | 99.3 |
| 3 kDa flow-through | 86.8 |

That what is claimed:

1. A skin care composition for use in the treatment of a scalp disorder, comprising an effective amount of a Bacillus velezensis fermentate, and/or metabolite thereof, wherein said composition reduces and/or treats said scalp disorder.

2. The skin care composition of claim 1, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

3. The skin care composition of claim 1, further comprising one or more antidandruff active agents.

4. The skin care composition of claim 1, wherein the composition reduces the growth of Staphylococcus species and/or Malassezia species.

5. The skin care composition of claim 1, wherein the composition removes biofilm of Malassezia species and/or Staphylococcus species.

6. The skin care composition of claim 1, further comprising an additional compound selected from the group consisting of an excipient, a preservative, a pH adjuster.

7. Use of an effective amount of the skin care composition of any preceding claim in a skin care product.

8. A skin care product comprising an effective amount of the skin care composition of any one of claim 1-6 and one or more dermatologically or skin care acceptable component.

9. The skin care product of claim 8, wherein said effective amount of the skin care composition is at least about 1%, 2%, 3%, 4% up to 5% on a weight basis relative to a total weight of said skin care product.

10. The skin care product of claim 8, wherein the product is formulated for topical administration.

11. A method for treating a scalp disorder in a subject in need thereof, comprising administering an effective amount of a Bacillus velezensis fermentate, and/or metabolite thereof, to said subject.

12. The method according to claim 11, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

13. The method according to claim 11, wherein the fermentate is administered topically.

14. A method for treating and/or reducing a dandruff condition of the scalp in a subject in need thereof, comprising administering an effective amount of a Bacillus velezensis fermentate, and/or metabolite thereof, to said subject.

15. The method according to claim 14, wherein the fermentate, and/or metabolite thereof, is administered topically.

16. The method of claim 14, wherein the composition is administered to the subject's skin or scalp.

17. The method of claim 14, wherein the fermentate, and/or metabolite thereof, reduces the growth of Staphylococcus species and/or Malassezia species.

18. The method of claim 14, wherein the fermentate, and/or metabolite thereof, removes biofilm of Malassezia species and/or Staphylococcus species

19. A method for treating and/or reducing a dandruff condition of the scalp in a subject in need thereof, comprising topically administering a skin care product comprising a skin care composition of any one of claim 1-6 to said subject.

20. The method of claim 19, wherein the skin care product is a selected from the group consisting of a lotion, a serum, a jelly, a cream, a gel, an emulsion, a solid cosmetic, a mask, a patch, and a stick comprising at least 1%, 2%, 3%, 4% up to 5% of said skin care composition on a weight basis relative to a total weight of said skin care product.


SEQUENCE LISTING (8 sequences)

SEQ ID NO: 1 - DNA, 21 bp, artificial sequence
  primer 8F
agagtttgatymtcctggctc

SEQ ID NO: 2 - DNA, 20 bp, artificial sequence
  primer 1492R
cggttaccttgttacgactt

SEQ ID NO: 3 - DNA, 1400 bp, Bacillus velezensis
ggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcaracaaccatccggtattagccccggtttcccggagttatcccagtcttacaggcaggttacccacgtgttactcacccgtccgccgctaacatcagggagcaagctcccatctgtccgctcgactgca

SEQ ID NO: 4 - DNA, 1419 bp, Bacillus velezensis
tcggcggctggctcctaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcaracaaccatccggtattagccccggtttcccggagttatcccagtcttacaggcaggttacccacgtgttactcacccgtccgccgctaacatcagggagcaagctcccatctgtccgctcgactgca

SEQ ID NO: 5 - DNA, 1415 bp, Bacillus velezensis
ttcggcggctggctcctaaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcaracaaccatccggtattagccccggtttcccggagttatcccagtcttacaggcaggttacccacgtgttactcacccgtccgccgctaacatcagggagcaagctcccatctgtccgctcg

SEQ ID NO: 6 - RNA, 1265 nt, Bacillus velezensis
cgaugcguagccgaccugagagggugaucggccacacugggacugagacacggcccagacuccuacgggaggcagcaguagggaaucuuccgcaauggacgaaagucugacggagcaacgccgcgugagugaugaagguuuucggaucguaaagcucuguuguuagggaagaacaagugccguucaaauagggcggcaccuugacgguaccuaaccagaaagccacggcuaacuacgugccagcagccgcgguaauacguagguggcaagcguuguccggaauuauugggcguaaagggcucgcaggcgguuucuuaagucugaugugaaagcccccggcucaaccggggagggucauuggaaacuggggaacuugagugcagaagaggagaguggaauuccacguguagcggugaaaugcguagagauguggaggaacaccaguggcgaaggcgacucucuggucuguaacugacgcugaggagcgaaagcguggggagcgaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacgggggcccgcacaagcgguggagcaugugguuuaauucgaagcaacgcgaagaaccuuaccaggucuugacauccucugacaauccuagagauaggacguccccuucgggggcagagugacagguggugcaugguugucgucagcucgugucgugagauguuggguuaagucccgcaacgagcgcaacccuugaucuuaguugccagcauucaguugggcacucuaaggugacugccggugacaaaccggaggaagguggggaugacgucaaaucaucaugccccuuaugaccugggcuacacacgugcuacaauggacagaacaaagggcagcgaaaccgcgagguuaagccaaucccacaaaucuguucucaguucggaucgcagucugcaacucgacugcgugaagcuggaaucgcuaguaaucgcggaucagcaugccgcggugaauacguucccgggccuuguacacaccgcccgucacaccacgagaguuuguaacacccgaagucggugagguaaccuuuuaggagccagccgccgaaggugggacagaugauuggggugaagucguaacaagguagccguaucggaaggugcggcuggaucaccuccuuu

SEQ ID NO: 7 - RNA, 1265 nt, Bacillus velezensis; identical to SEQ ID NO: 6

SEQ ID NO: 8 - RNA, 1265 nt, Bacillus velezensis; identical to SEQ ID NO: 6